(19) United States
(12) Patent Application Publication
(10) Pub. No.: US 2024/0001199 A1
(43) Pub. Date: Jan. 4, 2024

(54) TOTAL WELLNESS PLATFORM

(71) Applicants: RYAN SMALL, FOSTER, RI (US); ANTHONY ZONFRILLI, HOPE VALLEY, RI (US); JAMES FRANK, LINCOLN CITY, OR (US); KATE MACINANTI, KINGSTON, RI (US); TODD DAVIS, PHOENIX, AZ (US)

(72) Inventors: RYAN SMALL, FOSTER, RI (US); ANTHONY ZONFRILLI, HOPE VALLEY, RI (US); JAMES FRANK, LINCOLN CITY, OR (US); KATE MACINANTI, KINGSTON, RI (US); TODD DAVIS, PHOENIX, AZ (US)

(21) Appl. No.: 17/985,083

(22) Filed: Nov. 10, 2022

Related U.S. Application Data
(60) Provisional application No. 63/278,166, filed on Nov. 11, 2021.

(51) Int. Cl. A63B 24/00; G16H 50/20; G16H 20/30; G16H 20/60; G16H 20/70; G05B 15/02

(52) U.S. Cl. CPC A63B 24/0075; G16H 50/20; G16H 20/30; G16H 20/60; G16H 20/70; G05B 15/02

(57) ABSTRACT

A wellness platform for user fitness with a customizable, self-contained gym space having one or more physical training equipment assemblies. The physical training equipment assemblies may be conventional gym equipment and may also be equipment with health monitors which may further be operationally coupled to user computerized devices. The self-contained gym space and the physical training equipment are designed to employ one or more on-platform activity sensors, operationally coupled to at least one computer system. One or more activity recommendation systems are designed to recommend physical training equipment assemblies, nutrition, and psychological exercises and are designed to present wellness platform data, the data that may be used with machine learning systems, the machine learning systems designed to assess past and present performance and recommend future activities and targets. Off-platform sensors may be monitored. Environmental controls may be activated. Added are aquatic training systems which include pools.

### CLAIM OF PRIORITY

This application claims the benefit of and priority to U.S. provisional application with Ser. No. 63/278,166, filed on Nov. 11, 2021, which is incorporated herein by reference in its entirety.

### FIELD OF THE INVENTION

The inventive concept relates generally to a total wellness platform for mind and body health.

### BACKGROUND

Currently, there is no product-agnostic wellness marketplace to help customers evaluate products and methodologies in the health and wellness space. No widely available process considers the mind, nutrition, fitness, and personality factors for wellness. No customizable gym space comes connected to a network of customers and practitioners across a multitude of products and methodologies. There are many private networks associated with a specific product or methodology such as Nutrisystem, Isogenix, Pelaton, Noom, and others that build a community of users of their products.

People need help putting a plan together to achieve wellness goals, decide on products, and select training methodologies. Current solutions lack an agnostic platform that allows them to connect with others in a similar situation to recommended activities, products, and practitioners. Therefore, there is a need for an improved training platform for mind and body health.

### SUMMARY OF THE INVENTION

Disclose is a wellness platform for user fitness with a customizable, self-contained gym space having one or more physical training equipment assemblies from a group of: cardio training equipment, flexibility training equipment, strength training equipment, balance training equipment, and coordination training equipment. The physical training equipment assemblies may be conventional gym equipment and may also be equipment with health monitors, the health monitors which may further be operationally coupled to user computerized devices such as a smartphone or smartwatch. The health monitors may monitor such health factors as pulse, temperature, calories burned, brainwaves, respiration, and other measures.

The self-contained gym space and the physical training equipment assemblies are further designed to employ one or more on-platform activity sensor assemblies from a group of: digital cameras, optical sensors, accelerometers, global positioning systems, gyroscopes, solid state compasses, pressure sensors, radio-frequency and identification tags, repetition counters, resistance monitors, timers, distance calculators, speedometers, trajectory sensors, proximity sensors, and calorie measures, the platform activity sensors which may further be operationally coupled to one or more user computerized devices such as a smartphone or smartwatch. The optical sensors include one or more of visible light spectrum sensors and infrared sensors. In some embodiments, the infrared sensors may be thermographic sensors.

In one embodiment of the wellness platform for user fitness, the physical training equipment assemblies are from a group of one or more of: mechanical fixed form, mechanical dynamic, mechanical electrical, and electrical virtual.

In one embodiment of the wellness platform for user fitness, one or more performance tracking systems are presented on one or more from a group of: a wearable user interface and a non-wearable user interface.

In one embodiment of the wellness platform for user fitness, one or more activity recommendation systems are presented on one or more from a group of: at least one wearable user interface and at least one non-wearable user interface.

In one embodiment of the wellness platform for user fitness, the one or more activity recommendation systems are designed to recommend activities involving one or more of physical training equipment assemblies, nutrition, and psychological exercises. Such activity recommendation systems may include such models as the Myers Brigg psychology system.

In one embodiment of the wellness platform for user fitness, the one or more activity recommendation systems draws assessable wellness platform data from one or more of user profile data, user past performance data, user present performance data, and forecasted user future performance data, the forecasted user future performance data which may further include goal thresholds.

In one embodiment of the wellness platform for user fitness, the assessable wellness platform data is assessed by one or more machine learning software systems from a group of: decision trees, neural networks, Bayesian networks, genetic programs, and nearest neighbor. Other machine learning algorithms may be used.

In one embodiment of the wellness platform for user fitness, the performance tracking system and the one or more activity recommendation systems are operationally coupled to one or more computer systems having a computer processing unit and a memory storage medium.

In one embodiment of the wellness platform for user fitness, the one or more performance tracking systems, one or more activity recommendation systems, and one or more computer systems are further operationally coupled to one or more wireless receiver and transmitter systems.

In one embodiment of the wellness platform for user fitness, the one or more computer systems are further designed to receive user data from the physical training assemblies, the performance tracking systems, and data from outside the wellness platform, the data from outside the wellness platform including one or more of user input and input from one or more off-platform activity sensors. Such off-platform activity sensors may include such items as a personal cardio monitor, a sleep monitor, and a calorie intake monitor.

In one embodiment of the wellness platform for user fitness, one or more environmental control systems are operationally coupled to one or more computer systems and are designed to control a setting from one or more of sound, light, odor, temperature, humidity, and airflow, the setting set from one or more of: user commands, user location on the platform, time, engagement in an activity measured by the performance tracking system, and engagement in an activity recommended by the activity recommendation system.

The inventive concept may be one or more of app-based, Web based, and computer system based.

In one embodiment of the wellness platform for user fitness, the self-contained space is made from at least one cargo shipping container.

In one embodiment of the wellness platform, a coaching interface allows a coach to monitor the activities of one or more users and provide both recommendations and encouragement.

The inventive concept may employ artificial intelligence “AI”, the concept of creating intelligent machines that can simulate at least some aspects of human thinking capability and behavior. AI is a subset of computer science and data science, and AI includes machine learning. Many AI applications improve operational performance of systems and may serve as decision support tools for users and for people such as coaches assisting users. AI will proficiently help users 1) assess their training needs, 2) make training decisions, 3) take training actions, 4) assess results, and so on from 5) a framework associated with best training practice and user goals. Machine learning helps AI to improve performance with varying ranges of human and machine data input. AI may be adjusted to account for best practices and the available equipment to make the best use of the resources at a given wellness platform.

Added is a representative embodiment that include aquatic equipment. This representative embodiment includes a wellness platform for user fitness that has a customizable, self-contained fitness space having at least one or more physical training equipment assemblies from a group of: cardio training equipment, flexibility training equipment, strength training equipment, balance training equipment, aquatic training equipment, a hot tub, and coordination training equipment. The self-contained fitness space and the physical training equipment assemblies further designed to employ one or more on-platform activity sensor assemblies from a group of: digital cameras, optical sensors, accelerometers, global positioning systems, gyroscopes, solid state compasses, pressure sensors, radio-frequency and identification tags, repetition counters, resistance monitors, timers, distance calculators, speedometers, trajectory sensors, proximity sensors, waterflow sensors, and calorie measures.

The optical sensors include at least one or more of visible light spectrum sensors and infrared sensors. The physical training equipment assemblies from a group of at least one or more of: mechanical fixed form, mechanical dynamic, mechanical electrical, and electrical virtual. At least one or more performance tracking systems presented on one or more from a group of: a wearable user interface and a non-wearable user interface. At least one or more activity recommendation systems presented on one or more from a group of: at least one wearable user interface and at least one non-wearable user interface. The at least one or more activity recommendation systems is designed to recommend activities involving at least one or more of the at least one or more physical training equipment assemblies, nutrition, and psychological exercises.

This added embodiment of the wellness platform for user fitness has at least one environmental control system operationally coupled to the at least one computer system adapted to control a setting from at least one or more of sound, light color, light tone, light periodicity, odor, air temperature, water temperature, humidity, airflow, waterflow, and waterjet flow the setting set from at least one or more of a user command, user location on the platform, time, engagement in an activity measured by the performance tracking system, and engagement in an activity recommended by the activity recommendation system, the assembled wellness platform data assessed via at least one or more machine learning software systems from a group of: decision trees, neural networks, Bayesian networks, genetic programs, and nearest neighbor. The aquatic space may be disposed within at least one cargo shipping container. Periodicity is variability such as a light being periodically on and off or having some quality such as brightness or color that changes over time.

The inventive concept now will be described more fully hereinafter with reference to the accompanying drawings, which are intended to be read in conjunction with both this summary, the detailed description, and any preferred and/or particular embodiments specifically discussed or otherwise disclosed. This inventive concept may, however, be embodied in many different forms and should not be construed as limited to the embodiments set forth herein; rather, these embodiments are provided by way of illustration only and so that this disclosure will be thorough, complete, and will fully convey the full scope of the inventive concept to those skilled in the art.

### BRIEF DESCRIPTION OF THE DRAWINGS

FIG. 1 illustrates components of the disclosed wellness platform for user fitness;

FIG. 2 illustrates an exemplary interior view of a representative wellness platform for user fitness;

FIG. 3 illustrates a representative shipping container;

FIG. 4A illustrates physical training assemblies;

FIG. 4B illustrates on-platform activity sensor assemblies;

FIG. 4C illustrates light sensors;

FIG. 4D illustrates a smartphone and smartwatch;

FIG. 4E illustrates physical training assemblies;

FIG. 4F illustrates a wearable and non-wearable interface;

FIG. 4G illustrates physical, nutritional, and psychological exercises;

FIG. 4H illustrates drawn data types;

FIG. 4I illustrates machine learning systems;

FIG. 4J illustrates environmental control systems;

FIG. 5 illustrates a representative computer system;

FIG. 6A illustrates a representative method for using the wellness platform;

FIG. 6B further illustrates the representative method for using the wellness platform;

FIG. 7 illustrates a representative coaching interface;

FIGS. 8A-8B illustrate representative avatars and statistics;

FIGS. 9A-9B illustrates a representative wellness platform method;

FIGS. 10A-10D Illustrates aquatic training equipment modules; and

FIG. 11 illustrates a fitness system including aquatic training equipment represented as a pool.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

The inventive concept is a wellness platform and marketplace that spans the mind, nutrition, and fitness aspects of health. It consists of a software application and a physical gym space that are designed to work together but can exist separately. Novel aspects are a combination of 1) mind, 2) nutrition, 3) fitness, and 4) psychological-based systems—such as Myers-Briggs-based personality type—to create a custom wellness plan for individuals and to help connect individuals with practitioners and other users in a similar disposition to support the success of their wellness goals. The inventive concept uses proprietary matching algorithms that factor in health and personality to recommend appropriate wellness methodologies, disciplines, and practitioners of those methodologies. The inventive concept uses science standards to vet products and methodologies for acceptance and recommendations. Software types inherent in the inventive concept may include such external third-party peripherals as Apple watch, Fitbit, and Halo wristband. The physical gym space features adjustable lighting and sound, adjustments based on the type of exercise or activity being performed.

The inventive concept can establish a user profile and recommend types of activities users should consider based on their profile. The inventive concept can be a platform for products and practitioners to have a social media-based marketplace where a plurality of products can exist and be found and evaluated on a single platform. The inventive concept can allow practitioners of methodologies and products to bundle their offerings for a customized wellness plan. By having accountability partners and coaches with a software application that will recommend new activities if what the user is trying is not working as tracked by the software application.

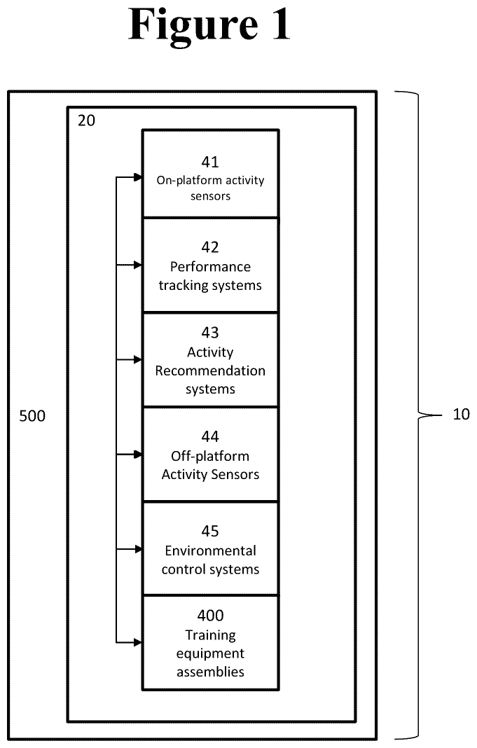

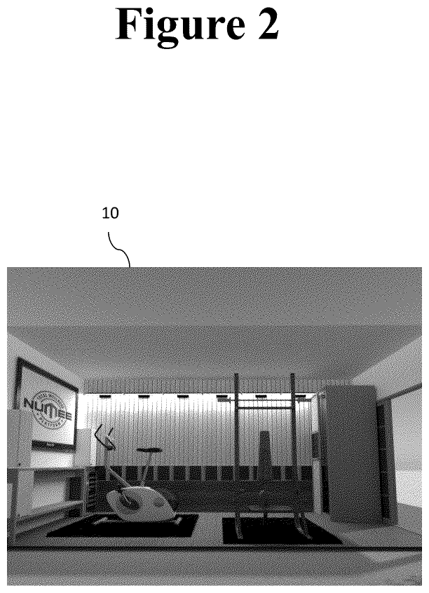

FIGS. 1 and 2 illustrates an exemplary embodiment controlled by at least one computer system 500, through which operationally couples components of a wellness platform 10 including a customizable, a self-contained space 20, one or more physical training equipment assemblies 400, one or more on-platform activity sensors 41, one or more performance tracking systems 42, one or more activity recommendation systems 43, one or more off-platform activity sensors 44, and environmental control systems 45.

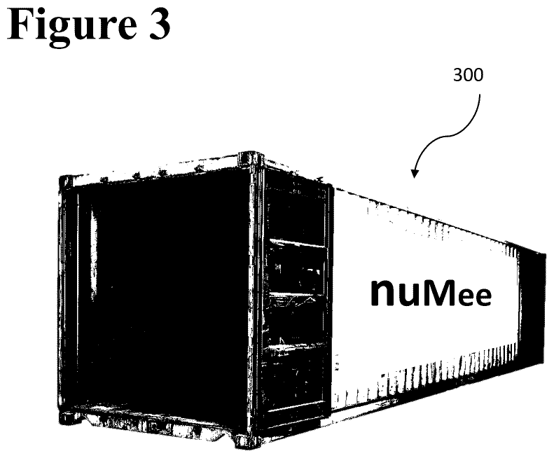

FIG. 3 illustrates that in one embodiment of the wellness platform, the self-contained space is made from at least one cargo shipping container 300. The inventive concept may use common sizes of shipping containers sold in North America: 20-foot and 40-foot units. The measurement is the length of the unit. All containers transported by cargo ship need to be a fixed width of 8 feet. The inventive concept may also use 53-foot containers made to rail standards and may use other container systems, including bespoke container systems. Other container sizes may be used along with modular construction units with rectangular, container-like, frames. Such containers may be wider than shipping containers.

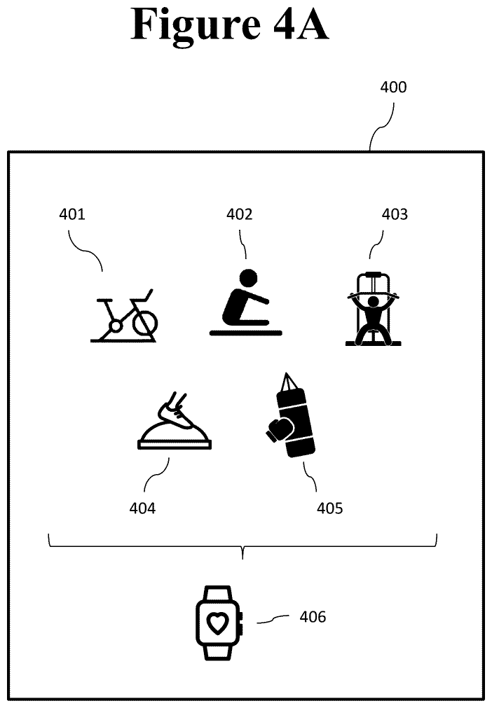

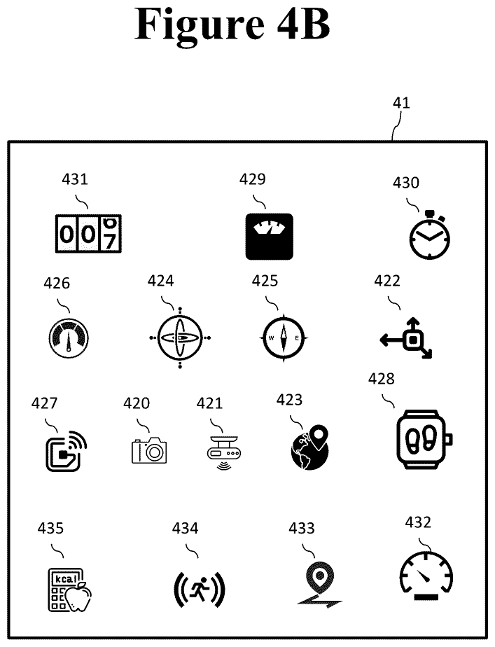

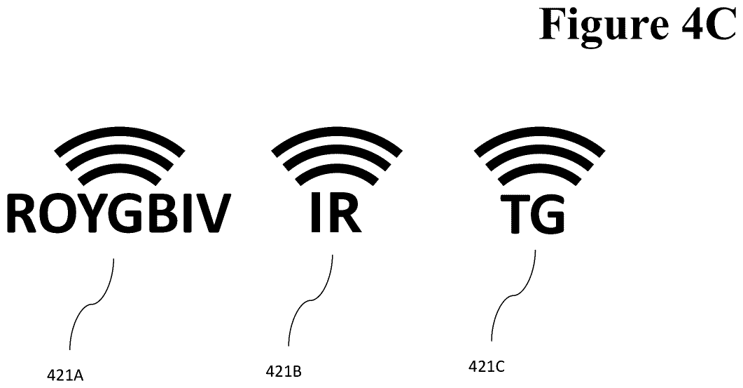

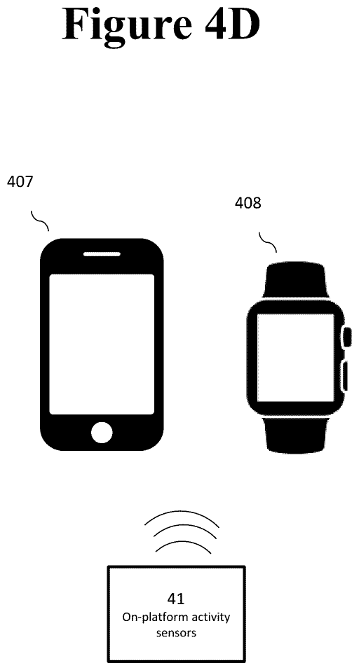

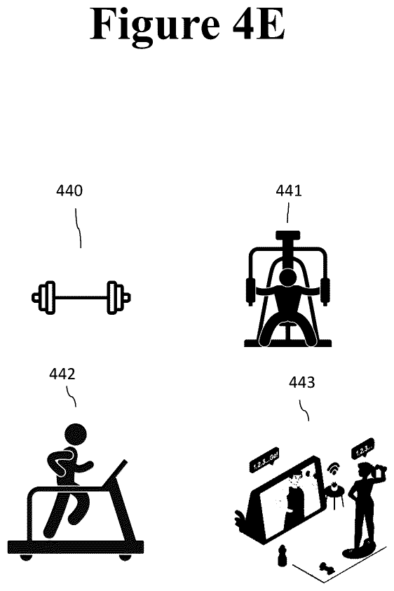

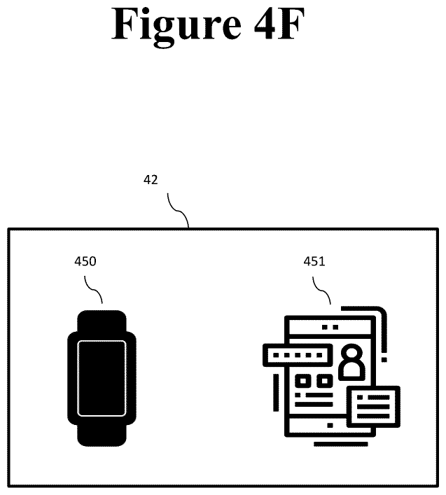

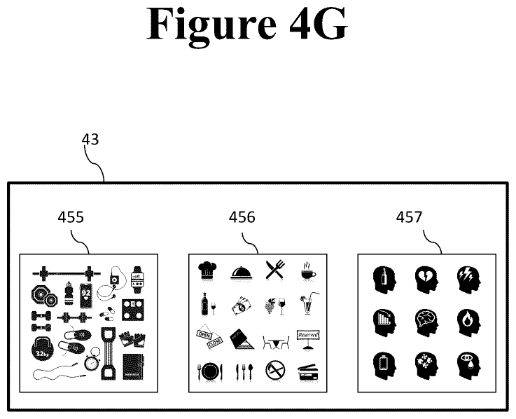

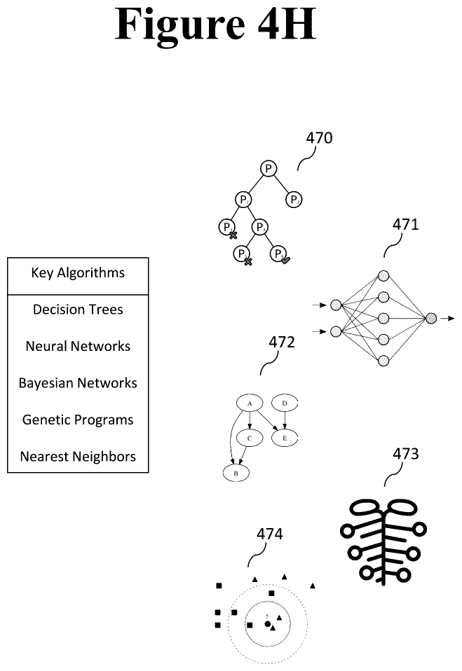

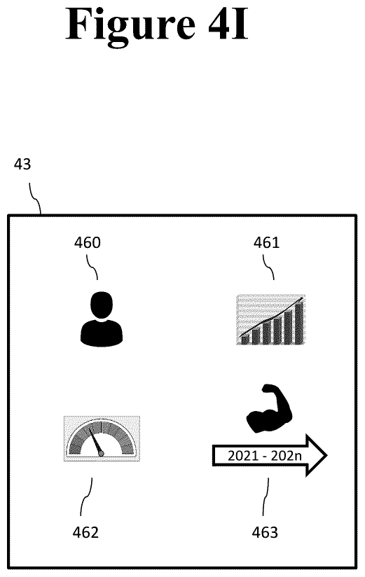

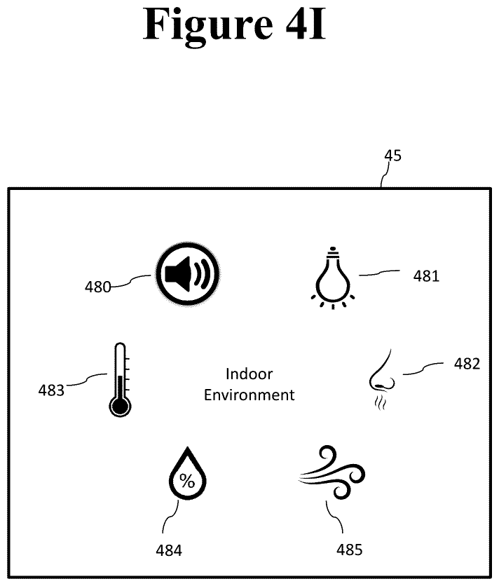

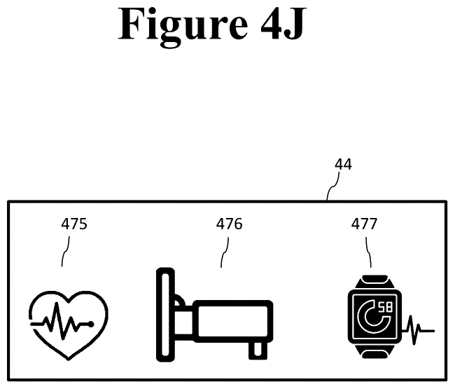

FIG. 4A illustrates that in this exemplary embodiment, wellness platform 10 for user fitness has within or in proximity of the customizable, self-contained gym space 20, one or more physical training equipment assemblies 400 from a group of: cardio training equipment 401, flexibility training equipment 402, strength training equipment 403, balance training equipment 404, and coordination training equipment 405. The physical training equipment assemblies may be conventional gym equipment and may also be equipment with health monitors 406, health monitors 406 which may further be operationally coupled to user computerized devices such as, as illustrated in FIG. 4D, a smartphone 407 or smartwatch 408. Health monitors 406 may monitor such health factors as pulse, temperature, calories burned, brainwaves, respiration, and other measures.

FIG. 4B illustrates that self-contained gym space 20 and physical training equipment assemblies 400 are further designed to employ one or more on-platform activity sensor assemblies 41 from a group of: digital cameras 420, optical sensors 421, accelerometers 422, global positioning systems 423, gyroscopes 424, solid state compasses 425, pressure sensors 426, radio-frequency and identification tags 427, repetition counters 428, resistance monitors 429, timers 430, distance calculators 431, speedometers 432, trajectory sensors 433, proximity sensors 434, and calorie measures 435, the platform activity sensors 41 which may further be operationally coupled to one or more user computerized devices such as smartphone 407 or smartwatch 408. Optical sensors 421 include, as illustrated in FIG. 4C, one or more of visible light spectrum sensors 421A and infrared sensors 421B. In some embodiments, the infrared sensors may be thermographic sensors 421C.

FIG. 4E illustrates physical training equipment assemblies 400 are from a group of one or more of: mechanical fixed form 440, mechanical dynamic 441, mechanical electrical 442, and electrical virtual 443.

FIG. 4F illustrates that the one or more performance tracking systems 42 are presented on one or more of a group of: a wearable user interface 450 and a non-wearable user interface 451. The one or more activity recommendation systems 43 are presented on one or more of the group of: at least one wearable user interface 450 and at least one non-wearable user interface 451.

FIG. 4G illustrates that the one or more activity recommendation systems 43 are designed to recommend activities involving one or more of physical training equipment assemblies 455, nutrition 456, and psychological exercises 457. Such recommendation systems may include such models as the Myers-Brigg psychology system.

FIG. 4H illustrates that the one or more activity recommendation systems 43 draws assessable wellness platform data from one or more of user profile data 460, user past performance data 461, user present performance data 462, and forecasted user future performance data 463, forecasted user future performance data 463 which may further include goal thresholds.

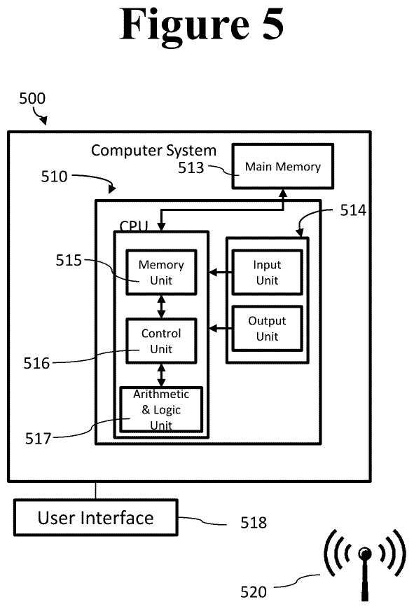

FIG. 4I illustrates the assessable wellness platform data is assessed via one or more machine learning software systems from a group of: decision trees 470, neural networks 471, Bayesian networks 472, genetic programs 473, and nearest neighbor 474. The performance tracking system and the one or more activity recommendation systems are operationally coupled with reference to FIG. 5, to at least one or more computer systems 500 having a computer processing unit 510 and a memory storage medium 513.

FIG. 4J illustrates that the one or more environmental control systems 45 are operationally coupled to the at least one or more computer systems 500 and are designed to control a setting from one or more of sound 480, light 481, odor 482, temperature 483, humidity 484, and airflow 485, the setting set from one or more of: user command, user location on the platform, time, engagement in an activity measured by the performance tracking system, and engagement in an activity recommended by the activity recommendation system.

FIG. 5 illustrates the one or more performance tracking systems 42, one or more activity recommendation systems 43, and the one or more computer systems 500 are further operationally coupled to one or more wireless receiver and transmitter systems 520. The one or more computer systems 500 are further designed to receive user data from physical training assemblies 400, performance tracking systems 42, and data from outside the wellness platform 10, data from outside the wellness platform 10 including one or more of user input and input from the one or more off-platform activity sensors 44. Such off-platform activity sensors 44 may include such items as a personal cardio monitor 475, a sleep monitor 476, and a calorie intake monitor 477.

FIG. 5 further illustrates an exemplary computer processor central processing unit (CPU) 510, also called a central processor or main processor, which is the electronic circuitry within the representative at least one computer 500 that executes instructions that make up a computer program. CPU 510 performs basic arithmetic, logic, controlling, and input/output (I/O) operations specified by the instructions in the program. Arithmetic and logic unit (ALU) 517 is a combination digital electronic circuit that performs arithmetic and bitwise operations in integer binary numbers. Traditionally, the term CPU refers to a processor, more specifically to its processing unit and control unit (CU) 516, distinguishing these core elements of a computer from external components such as main memory 513 and input output (I/O) circuitry 514. CPU 510 may also contain memory 515. Memory refers to a component that is used to store data for immediate use in a computer. A user interface 518 is illustrated on this representative embodiment operationally coupled to central processing unit (CPU) 510 having memory unit 515.

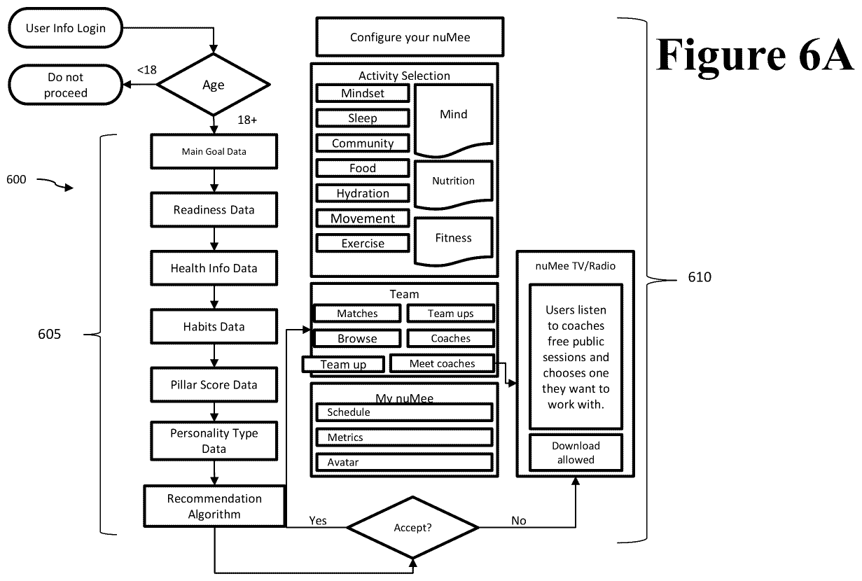

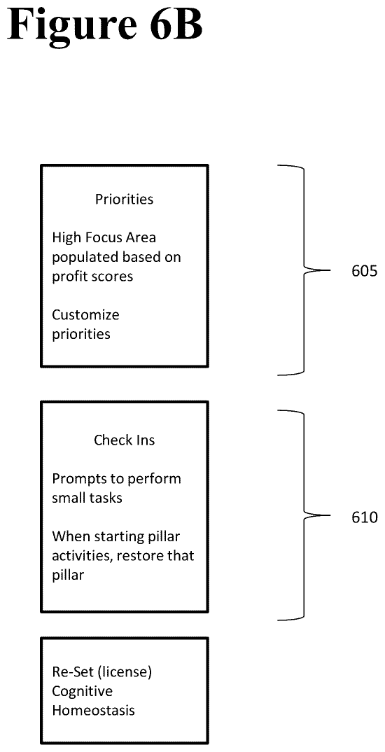

FIGS. 6A and 6B illustrates that in this exemplary embodiment, a method 600 for using wellness platform 10. The method includes assessment phase 605 and recommendation phase 610.

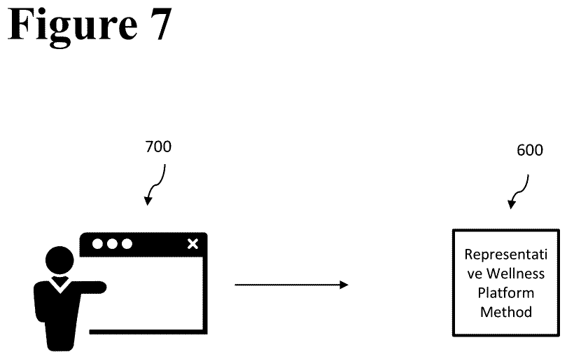

FIG. 7 illustrates one embodiment of wellness platform 10 wherein a coaching interface 700 allows a coach to monitor the activities of one or more users and provide both recommendations and encouragement. Coach can also mean trainer.

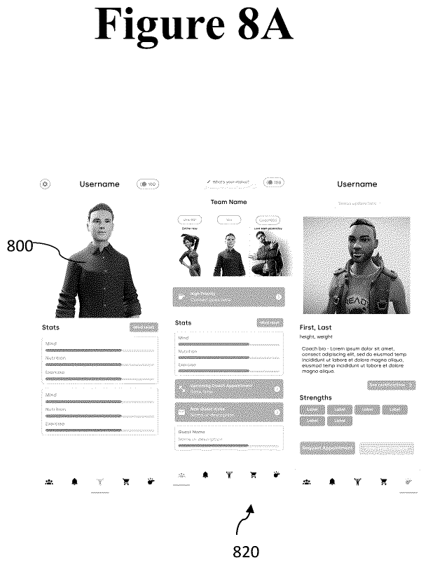

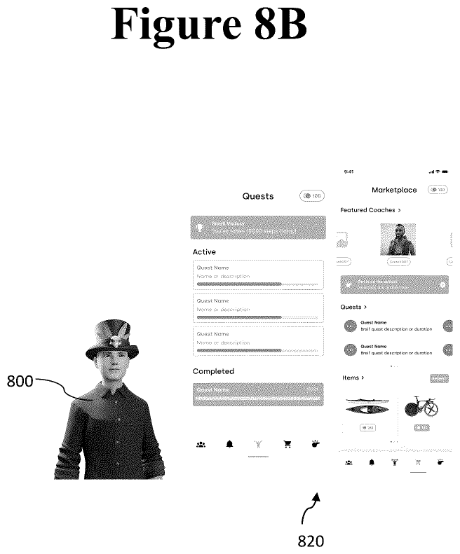

FIGS. 8A-8B illustrate that the inventive concept may use an avatar 800 that the user creates and that reflects their efforts in the real world. Avatar 800 may be improved and advanced by the user's real-world efforts.

One method for using the wellness platform includes the user logging onto wellness platform 10. Documents and a subdomain aid the user in creating and setting up personal avatar 800. In this representative embodiment, the user will create their personal avatar on an avatar setup. Upon creating the personal avatar, the user will send the personal avatar to their user profile. Once the personal avatar is complete, the personal avatar is sent to the user's profile on the application. The application is substantially a platform that may be bespoke or may be disposed on a smart TV or other visualizing device, the user able to decide on the platform. Upon entering the platform, the user can go to a wellness profile which presents a questionnaire guide for next steps. The next steps provide substantially a framework for a user experience that leads them to their user profile and dashboard 820. The user experience will include mindset, nutrition, and physical exercises and may further lead to a marketplace for mindset, nutrition, and physical fitness products and services. The user experience may further lead to educational products and services associated with the mindset, nutrition, and physical fitness products and services and may be oriented towards children as well as adults.

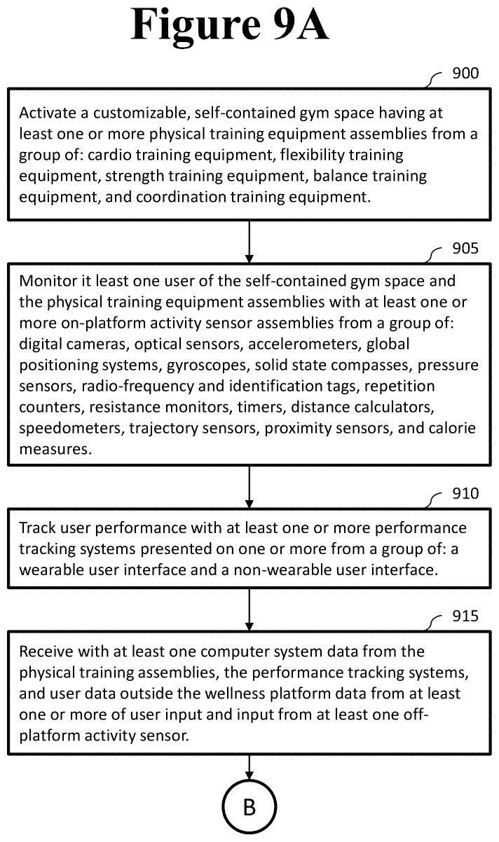

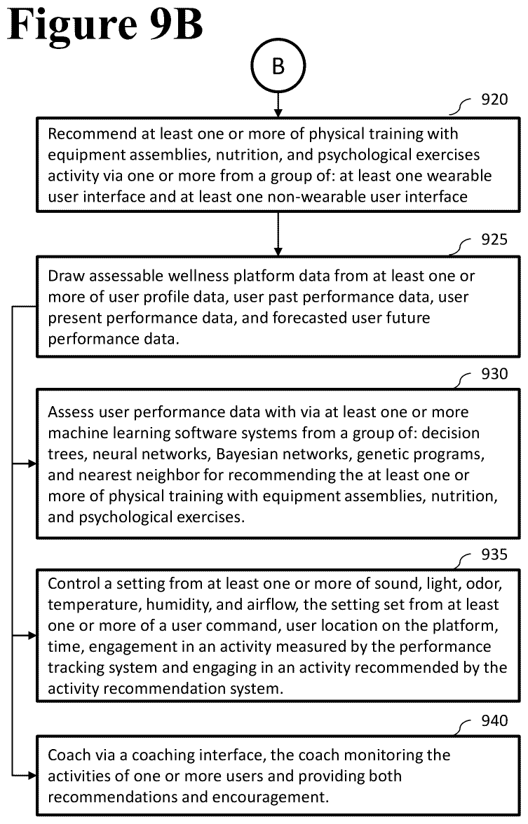

FIGS. 9A-9B illustrate a wellness platform method for user fitness, the method including the step of 900, activating a customizable, self-contained gym space 20 having at least one or more physical training equipment assemblies 400 from a group of: cardio training equipment 401, flexibility training equipment 402, strength training equipment 403, balance training equipment 404, and coordination training equipment 405. The method further includes the step of 905, monitoring it least one user of self-contained gym space 20 and physical training equipment assemblies 400 with at least one or more on-platform activity sensor assemblies from a group of: digital cameras, optical sensors 421, accelerometers 422, global positioning systems 423, gyroscopes 424, solid state compasses 425, pressure sensors 426, radio-frequency and identification tags 427, repetition counters 428, resistance monitors 429, timers 428, distance calculators 429, speedometers 430, trajectory sensors 433, proximity sensors 444, and calorie measures 445.

FIGS. 9A-9B further illustrate that the method includes the step of 910, tracking user performance with at least one or more performance tracking systems 42 presented on one or more from a group of: a wearable user interface 450 and a non-wearable user interface 451. The method further includes the step of 915, receiving with at least one computer system 500 data from physical training assemblies 400, performance tracking systems 42, and user data outside the wellness platform data from at least one or more of user input and input from at least one off-platform activity sensor 44. The method further includes the step of 920, recommending at least one or more of physical training with equipment assemblies, nutrition, and psychological exercises activity by way of one or more from a group of: at least one wearable user interface 450 and at least one non-wearable user interface 451. The method further includes the step of 925, drawing assessable wellness platform data from at least one or more of user profile data 460, user past performance data 461, user present performance data 462, and forecasted user future performance data 463, the forecasted user future performance data 463 including goal thresholds.

FIGS. 9A-9B illustrate that the wellness platform method may further include the step of 930, assessing user performance data 461, 462, 463 with via at least one or more machine learning software systems from a group of: decision trees 470, neural networks 471, Bayesian networks 472, genetic programs 473, and nearest neighbor 474 for recommending the at least one or more of physical training with equipment assemblies, nutrition, and psychological exercises.

FIGS. 9A-9B further illustrate that the wellness platform method may include the step of 935, controlling a setting from at least one or more of sound, light, odor, temperature, humidity, and airflow, the setting set from at least one or more of a user command, user location on the platform, time, engagement in an activity measured by the performance tracking system and engaging in an activity recommended by the activity recommendation system 43. The wellness platform method may further include the step of 940, coaching via coaching interface 700, the coach monitoring the activities of one or more users and providing both recommendations and encouragement.

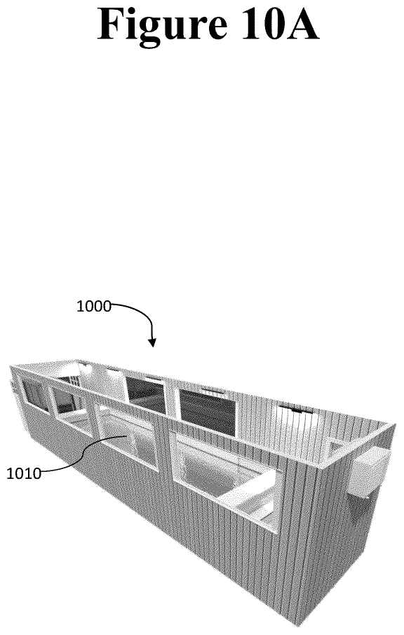

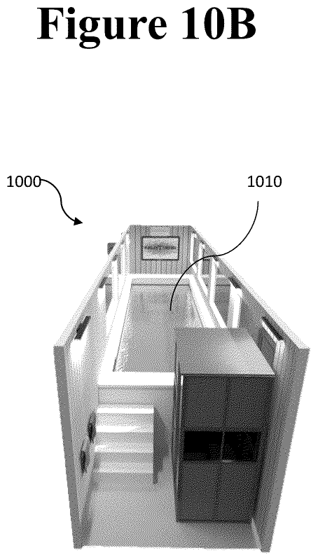

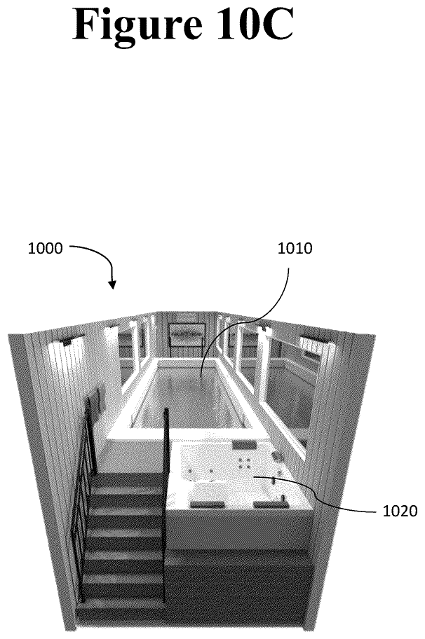

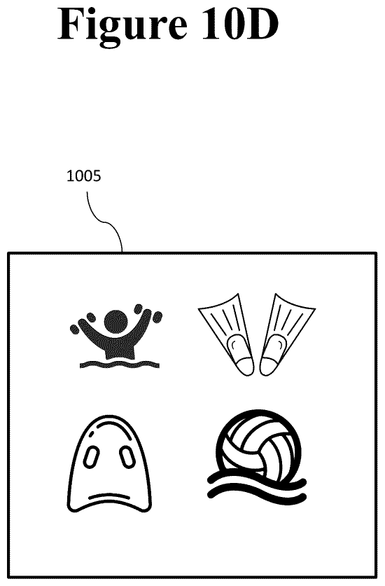

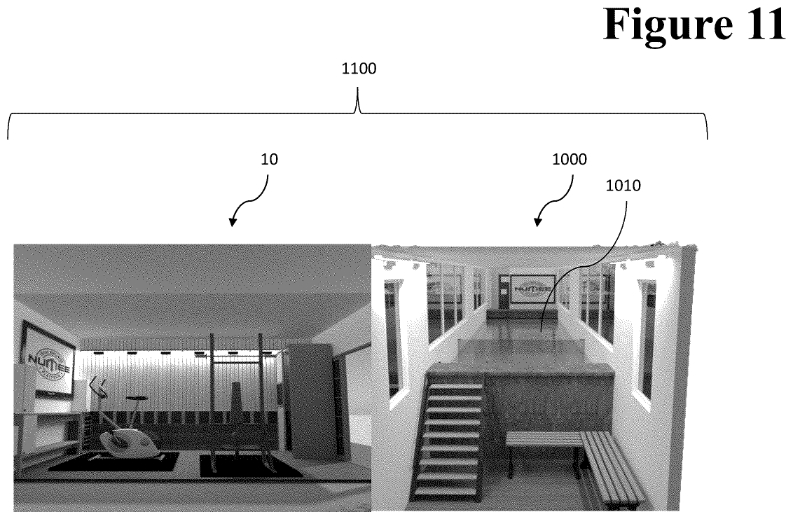

FIGS. 10A-10B illustrate an added representative embodiment that includes aquatic equipment 1005. This representative embodiment includes a wellness platform for user fitness that has a customizable, as illustrated in FIG. 11, a self-contained fitness space 1000 having at least one or more physical training equipment assemblies 400 from a group of: cardio training equipment 401, flexibility training equipment 402, strength training equipment 403, balance training equipment 404, aquatic training equipment 1005 which may include a pool 1010 that pool which may include flowing water, a hot tub 1020, and coordination training equipment 405. Aquatic training equipment 1005 may be used for programs inclusive of other equipment or may be used independently as part of aquatic systems 1000. Self-contained fitness space 1000 and physical training equipment assemblies 400 further designed to employ one or more on-platform activity sensor assemblies from a group of: digital cameras 420, optical sensors 421, accelerometers 422, global positioning systems 423, gyroscopes 424, solid state compasses 425, pressure sensors 426, radio-frequency and identification tags 427, repetition counters 428, resistance monitors 429, timers 430, distance calculators 431, speedometers 432, trajectory sensors 433, proximity sensors 434, waterflow sensors 436, and calorie measures 435.

This added embodiment of the wellness platform for user fitness has at least one environmental control system 45 operationally coupled to the at least one computer system 500 adapted to control a setting from at least one or more of sound, light color, light tone, light periodicity, odor, air temperature, water temperature, humidity, airflow, waterflow, and waterjet flow the setting set from at least one or more of a user command, user location on the platform, time, engagement in an activity measured by the performance tracking system, and engagement in an activity recommended by the activity recommendation system 43. The assembled wellness platform data may be assessed via at least one or more machine learning software systems from a group of: decision trees 470, neural networks 471, Bayesian networks 472, genetic programs 473, and nearest neighbor 474. The aquatic equipment 1005 may be disposed within at least one cargo shipping container 300.

A list of elements follows:
10 wellness platform
20 self-contained gym space
40 on-platform activity sensors
41 performance tracking system
42 activity recommendation systems
43 off-platform activity sensors
44 environmental control systems
300 cargo shipping container
400 physical training equipment assemblies
401 cardio training equipment
402 flexibility training equipment
403 strength training equipment
404 balance training equipment
405 coordination training equipment
406 health monitors
407 smartphone
408 Smartwatch
420 digital cameras
421 optical sensors
4211A visible light spectrum sensors
421B infrared sensors
421C thermographic sensors
422 Accelerometers
423 global positioning systems
424 Gyroscopes
425 solid state compasses
426 pressure sensors
427 radio-frequency and identification tags
428 repetition counters
429 resistance monitors
430 timers
431 distance calculators
432 speedometers
433 trajectory sensors
434 proximity sensors
435 calorie measures
436 waterflow sensors
440 mechanical fixed form
441 mechanical dynamic
442 mechanical electrical
443 electrical virtual
450 wearable user interface
451 non-wearable user interface
455 physical training equipment assemblies
456 nutrition
457 psychological exercises
461 profile data
461 past performance data
462 present performance data
463 forecasted user future performance data
470 decision trees
471 neural networks
472 Bayesian networks
473 genetic programs
474 nearest neighbor
475 cardio monitor
476 sleep monitor
477 calorie intake monitor
480 a sound
481 light
482 odor
483 temperature
484 humidity
485 airflow
500 computer systems
510 central processing unit CPU
513 memory storage medium
515 memory unit
514 input output (I/O) circuitry
517 arithmetic and logic units
518 user interface
520 wireless receiver and transmitter systems
700 coaching interface
1000 aquatic system
1005 aquatic training equipment
1010 pool
1020 hot tub
1100 fitness system

The following patents are incorporated by reference in their entireties: U.S. Pat. Nos. 5,761,854, 7,285,090, 8,038,577, 8,398,546, 9,165,117, 9,230,064, 9,750,999, 10,102,345, 10,133,849, 10,967,224, US2004034289, US2006205564, US2019378431, US20150308144, WO17177291, and WO19212369.

While the inventive concept has been described above in terms of specific embodiments, it is to be understood that the inventive concept is not limited to these disclosed embodiments. Upon reading the teachings of this disclosure, many modifications and other embodiments of the inventive concept will come to mind of those skilled in the art to which this inventive concept pertains, and which are intended to be and are covered by both this disclosure and the appended claims. It is indeed intended that the scope of the inventive concept should be determined by proper interpretation and construction of the appended claims and their legal equivalents, as understood by those of skill in the art relying upon the disclosure in this specification and the attached drawings.

1. A wellness platform for user fitness comprising:
a customizable, self-contained gym space having at least one or more physical training equipment assemblies from a group of: cardio training equipment, flexibility training equipment, strength training equipment, balance training equipment, and coordination training equipment;
the self-contained gym space and the physical training equipment assemblies further adapted to employ one or more on-platform activity sensor assemblies from a group of: digital cameras, optical sensors, accelerometers, global positioning systems, gyroscopes, solid state compasses, pressure sensors, radio-frequency and identification tags, repetition counters, resistance monitors, timers, distance calculators, speedometers, trajectory sensors, proximity sensors, and calorie measures;
the optical sensors including at least one or more of visible light spectrum sensors and infrared sensors;
the physical training equipment assemblies from a group of at least one or more of: mechanical fixed form, mechanical dynamic, mechanical electrical, and electrical virtual;
at least one or more performance tracking systems presented on one or more from a group of: a wearable user interface and a non-wearable user interface;
at least one or more activity recommendation systems presented on one or more from a group of: at least one wearable user interface and at least one non-wearable user interface;
the at least one or more activity recommendation systems adapted to recommend activities involving at least one or more of the at least one or more physical training equipment assemblies, nutrition, and psychological exercises.

2. The wellness platform for user fitness of claim 1 wherein the at least one or more activity recommendation systems draws assessable wellness platform data from at least one or more of user profile data, user past performance data, user present performance data, and forecasted user future performance data, the forecasted user future performance data which may further include goal thresholds.

3. The wellness platform for user fitness of claim 2 wherein the assessable wellness platform data is assessed via at least one or more machine learning software systems from a group of: decision trees, neural networks, Bayesian networks, genetic programs, and nearest neighbor.

4. The wellness platform for user fitness of claim 1 wherein the performance tracking system and the at least one or more activity recommendation systems are operationally coupled to at least one computer system having at least one computer processing unit, at least one memory storage medium, the at least one or more performance tracking systems, at least one or more activity recommendation systems, and at least one computer system further operationally coupled to at least one wireless receiver and transmitter system.

5. The wellness platform for user fitness of claim 4 wherein the at least one computer system is further adapted to receive data from the physical training assemblies, the performance tracking systems, and user data outside the wellness platform data from at least one or more of user input and input from at least one off-platform activity sensor.

6. The wellness platform for user fitness of claim 1 wherein the assessable wellness platform data is assessed via at least one or more machine learning software systems from a group of: decision trees, neural networks, Bayesian networks, genetic programs, and nearest neighbor.

7. The wellness platform of claim 1 wherein at least one environmental control system is operationally coupled to the at least one computer system adapted to control a setting from at least one or more of sound, light, odor, temperature, humidity, and airflow, the setting set from at least one or more of a user command, user location on the platform, time, engagement in an activity measured by the performance tracking system, and engagement in an activity recommended by the activity recommendation system.

8. The wellness platform for user fitness of claim 1 wherein the self-contained space is made from at least one cargo shipping container.

9. The wellness platform for user fitness of claim 1 wherein a coaching interface allows a coach to monitor the activities of one or more users and provide both recommendations and encouragement.

10. A wellness platform method for user fitness, the method comprising:
activating a customizable, self-contained gym space having at least one or more physical training equipment assemblies from a group of: cardio training equipment, flexibility training equipment, strength training equipment, balance training equipment, and coordination training equipment;
monitoring it least one user of the self-contained gym space and the physical training equipment assemblies with at least one or more on-platform activity sensor assemblies from a group of: digital cameras, optical sensors, accelerometers, global positioning systems, gyroscopes, solid state compasses, pressure sensors, radio-frequency and identification tags, repetition counters, resistance monitors, timers, distance calculators, speedometers, trajectory sensors, proximity sensors, and calorie measures;
tracking user performance with at least one or more performance tracking systems presented on one or more from a group of: a wearable user interface and a non-wearable user interface;
receiving with at least one computer system data from the physical training assemblies, the performance tracking systems, and user data outside the wellness platform data from at least one or more of user input and input from at least one off-platform activity sensor;
recommending at least one or more of physical training with equipment assemblies, nutrition, and psychological exercises activity by way of one or more from a group of: at least one wearable user interface and at least one non-wearable user interface;
drawing assessable wellness platform data from at least one or more of user profile data, user past performance data, user present performance data, and forecasted user future performance data; and
forecasting user future performance data to include goal thresholds.

11. The wellness platform method for user fitness of claim 10, the method further including assessing user performance data with at least one or more machine learning software systems from a group of: decision trees, neural networks, Bayesian networks, genetic programs, and nearest neighbor, and recommending the at least one or more of physical training with equipment assemblies, nutrition, and psychological exercises.

12. The wellness platform for user fitness of claim 10, the method further including controlling a setting from at least one or more of sound, light, odor, temperature, humidity, and airflow by at least one or more of a user command, user location on the platform, time, engagement in an activity measured by the performance tracking system, and engaging in an activity recommended by the activity recommendation system.

13. The wellness platform method for user fitness of claim 10, the method further including coaching via a coaching interface, the coach monitoring the activities of one or more users and providing both recommendations and encouragement.

14. A wellness platform for user fitness comprising:
a customizable, self-contained fitness space having at least one or more physical training equipment assemblies from a group of: cardio training equipment, flexibility training equipment, strength training equipment, balance training equipment, aquatic training equipment, a hot tub, and coordination training equipment;
the self-contained fitness space and the physical training equipment assemblies further adapted to employ one or more on-platform activity sensor assemblies from a group of: digital cameras, optical sensors, accelerometers, global positioning systems, gyroscopes, solid state compasses, pressure sensors, radio-frequency and identification tags, repetition counters, resistance monitors, timers, distance calculators, speedometers, trajectory sensors, proximity sensors, waterflow sensors, and calorie measures;
the optical sensors including at least one or more of visible light spectrum sensors and infrared sensors;
the physical training equipment assemblies from a group of at least one or more of: mechanical fixed form, mechanical dynamic, mechanical electrical, and electrical virtual;
at least one or more performance tracking systems presented on one or more from a group of: a wearable user interface and a non-wearable user interface;
at least one or more activity recommendation systems presented on one or more from a group of: at least one wearable user interface and at least one non-wearable user interface; and
the at least one or more activity recommendation systems adapted to recommend activities involving at least one or more of the at least one or more physical training equipment assemblies, nutrition, and psychological exercises.

15. The wellness platform for user fitness of claim 14 wherein the at least one or more activity recommendation systems draws assessable wellness platform data from at least one or more of user profile data, user past performance data, user present performance data, and forecasted user future performance data, the forecasted user future performance data which may further include goal thresholds.

16. The wellness platform for user fitness of claim 15 wherein the assessable wellness platform data is assessed via at least one or more machine learning software systems from a group of: decision trees, neural networks, Bayesian networks, genetic programs, and nearest neighbor.

17. The wellness platform for user fitness of claim 14 wherein the performance tracking system and the at least one or more activity recommendation systems are operationally coupled to at least one computer system having at least one computer processing unit, at least one memory storage medium, the at least one or more performance tracking systems, at least one or more activity recommendation systems, and at least one computer system further operationally coupled to at least one wireless receiver and transmitter system, a coaching interface further allowing a coach to monitor the activities of one or more users and provide both recommendations and encouragement.

18. The wellness platform for user fitness of claim 17 wherein the at least one computer system is further adapted to receive data from the physical training assemblies, the performance tracking systems, and user data outside the wellness platform data from at least one or more of user input and input from at least one off-platform activity sensor.

19. The wellness platform for user fitness of claim 14 wherein at least one environmental control system is operationally coupled to the at least one computer system and is adapted to control a setting from at least one or more of sound, light color, light tone, light periodicity, odor, air temperature, water temperature, humidity, airflow, waterflow, and waterjet flow the setting set from at least one or more of a user command, user location on the platform, time, engagement in an activity measured by the performance tracking system, and engagement in an activity recommended by the activity recommendation system.

20. The wellness platform for user fitness of claim 14 wherein the aquatic training equipment is disposed within at least one cargo shipping container.
